(19) United States
(12) Patent Application Publication
(10) Pub. No.: US 2023/0218339 A1
(43) Pub. Date: Jul. 13, 2023

(54) MEDICAL PUNCTURE DEVICE

(71) Applicant: Boston Scientific Medical Device Limited, Galway (IE)

(72) Inventors: Eduardo Moriyama, Richmond (CA); Ferryl Alley, Devonshire (BM); Kaylie Lau, Toronto (CA)

(21) Appl. No.: 18/188,133

(22) Filed: Mar. 22, 2023

Related U.S. Application Data
(63) Continuation of application No. PCT/IB2021/057601, filed on Aug. 18, 2021.
(60) Provisional application No. 63/081,369, filed on Sep. 22, 2020.

(51) Int. Cl. A61B 18/14; A61B 18/12

(52) U.S. Cl. CPC A61B 18/1487; A61B 18/1206; A61B 2018/00571

(57) ABSTRACT

A medical puncture device includes an elongate shaft having a proximal portion defining a proximal end and a distal portion defining a distal end. The distal portion tapers in outer diameter going towards the distal end to define a dilating tip. A lumen extends through the shaft from the proximal end to the distal end. The shaft includes a first electrical conductor that extends from the proximal portion to the distal portion and is electrically connectable to a radiofrequency generator. A radiofrequency puncture electrode is positioned proud of the distal end and is electrically connected to the first electrical conductor.

### CROSS REFERENCE TO RELATED APPLICATIONS

This application claims the benefit of International Application Number PCT/IB2021/057601, entitled “MEDICAL PUNCTURE DEVICE,” and filed Aug. 18, 2021, which claims the benefit of U.S. Provisional Application No. 63/081,369, entitled “MEDICAL PUNCTURE DEVICE,” and filed Sep. 22, 2020, which are hereby incorporated by reference in their entireties.

### FIELD

This document relates to medical devices. More specifically, this document relates to medical devices that use radiofrequency energy to puncture tissue.

### SUMMARY

The following summary is intended to introduce the reader to various aspects of the detailed description, but not to define or delimit any invention.

Medical puncture devices are disclosed. According to some aspects, a medical puncture device includes an elongate shaft having a proximal portion defining a proximal end and a distal portion defining a distal end. The distal portion tapers in outer diameter going towards the distal end to define a dilating tip. A lumen extends through the shaft from the proximal end to the distal end. The shaft includes a first electrical conductor that extends from the proximal portion to the distal portion and that is electrically connectable to a radiofrequency generator. A radiofrequency puncture electrode is positioned proud of the distal end and is electrically connected to the first electrical conductor.

In some examples, the shaft includes a polymeric sleeve, and the first electrical conductor is in the form of a metallic hypotube received in the polymeric sleeve. The polymeric sleeve can be a high-density polyethylene sleeve and the metallic hypotube can be a stainless steel hypotube.

In some examples, the shaft includes a polymeric inner layer and a polymeric outer layer, and the electrical conductor is positioned between the polymeric inner layer and the polymeric outer layer. The polymeric inner layer can be a high-density polyethylene inner layer, and the polymeric outer layer can be a low-density polyethylene layer.

In some examples, the electrical conductor is in the form of a wire or a braid.

In some examples, the device further includes a second electrical conductor electrically connecting the electrode to the first electrical conductor. The second electrical conductor can be in the form of a wire having a first end and a second end. The first end can be joined to the first electrical conductor, and the second end can be spaced distally of the distal end and joined to the radiofrequency puncture electrode. The wire can be J-shaped or coiled.

In some examples, the radiofrequency puncture electrode is retractable towards the shaft.

In some examples, the radiofrequency puncture electrode is atraumatic.

In some examples, the device includes a handle at the proximal end of the shaft. The first electrical conductor can be electrically connectable to the radiofrequency generator via the handle. The handle can include a hemostatic valve, a stopcock, and/or a syringe luer.

In some examples, the shaft has an outer diameter of between about 12.5 Fr and about 24 Fr.

In some examples, the electrode can collect electrical signals from the heart.

In some examples, the shaft is steerable.

In some examples, the distal portion is curved.

In some examples, the shaft includes a radiopaque marker, and/or an echogenic marker.

Medical methods are also disclosed. According to some aspects, a medical method includes advancing the medical puncture device towards an atrial septum; delivering radiofrequency energy from the radiofrequency puncture electrode to create a puncture in the atrial septum; and advancing the dilating tip through the puncture to dilate the puncture.

Medical puncture systems are also disclosed. According to some aspects, a medical puncture system includes a radiofrequency generator and a medical puncture device. The medical puncture device includes an elongate shaft having a proximal portion defining a proximal end and a distal portion defining a distal end. The distal portion tapers in outer diameter going towards the distal end to define a dilating tip. A lumen extends through the shaft from the proximal end to the distal end. The shaft includes a first electrical conductor extending from the proximal portion to the distal portion and electrically connectable to a radiofrequency generator. A radiofrequency puncture electrode is positioned proud of the distal end and electrically connected to the first electrical conductor.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical puncture system including a medical puncture device;

FIG. 2 is an enlarged view of the encircled region in FIG. 1;

FIG. 3 is a cross section taken through the medical puncture device of in FIG. 1;

FIG. 4A is a partial perspective view of the distal portion of another example medical puncture device, with the electrode thereof in a retracted configuration;

FIG. 4B is a partial perspective view of the distal portion of the medical puncture device of FIG. 4A, with the electrode thereof in a deployed configuration;

FIG. 5 is a partial perspective view of the distal portion of another example medical puncture device;

FIG. 6 is a partial perspective view of the distal portion of another example medical puncture device;

FIG. 7 is a schematic view of a step of a method for puncturing and dilating an atrial septum using the system of FIG. 1;

FIG. 8 is a schematic view of a subsequent step of the method of FIG. 7; and

FIG. 9 is a schematic view of a subsequent step of the method of FIG. 8.

### DETAILED DESCRIPTION:

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are medical devices that can be used to puncture and dilate tissue, and related systems and methods. Such devices may be referred to herein as ‘medical puncture devices’. The medical puncture devices may be used in a variety of medical procedures, but may be particularly useful in procedures that require transseptal access to the left atrium with a relatively large outer diameter catheter. Such procedures can include, for example, cryoablation procedures, mitral valve replacement procedures, and atrial appendage closure procedures. The medical puncture devices disclosed herein can puncture the atrial septum and dilate the puncture to a sufficiently large diameter (e.g. between about 12.5 Fr and about 24 Fr).

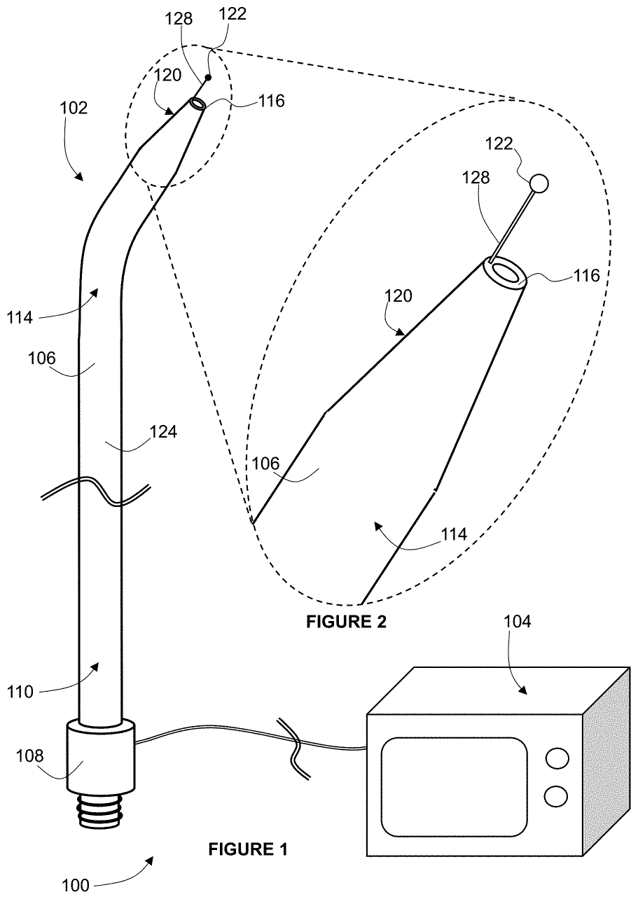

Referring now to FIGS. 1 and 2, a system 100 is shown that generally includes a medical puncture device 102, and a radiofrequency (RF) generator 104 to which the medical puncture device 102 is electrically connected.

The RF generator 104 can be any RF generator suitable for use in puncturing tissue, such as one sold by Baylis Medical Company (Montreal, Canada) under the brand name RFP-100A RF Puncture Generator, and/or other electrosurgery equipment, and will not be described in detail herein.

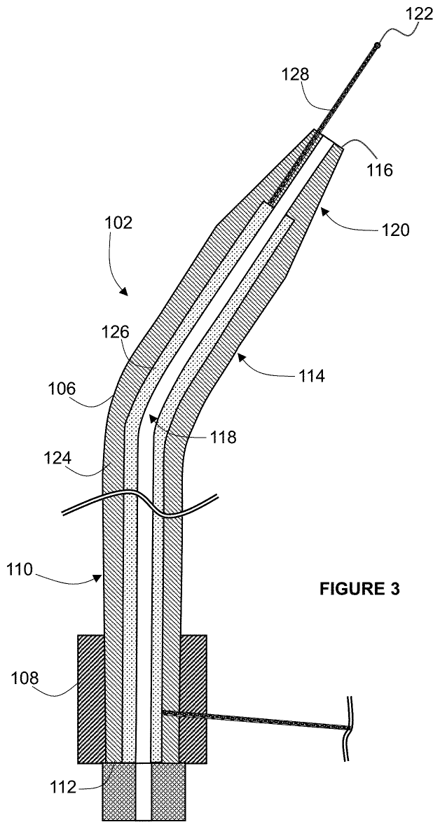

Referring still to FIGS. 1 and 2, as mentioned above, the medical puncture device 102 is configured to puncture tissue using RF energy, and to dilate the puncture to a relatively large diameter. In the example shown, the medical puncture device 102 includes an elongate shaft 106, a handle 108, and a radiofrequency puncture electrode 122. The shaft 106 has a proximal portion 110 that defines a proximal end 112 (shown in FIG. 3), and a distal portion 114 that defines a distal end 116. A lumen 118 (shown in FIG. 3) extends through the shaft 106 from the proximal end 112 to the distal end 116. The distal portion 114 is curved, and in the distal portion 114, the shaft 106 tapers in outer diameter going towards the distal end 116, to define a dilating tip 120. The handle 108 is at the proximal end 112, and can include various optional features such as a syringe luer, a hemostatic valve, and/or a stopcock (e.g. a 3-way stopcock) for fluid delivery through the lumen 118 via the handle 108.

As mentioned above, the shaft 106 can have a relatively large outer diameter, for example between about 12.5 Fr and about 24 Fr.

In the example shown, the shaft 106 is of a generally fixed shape. In alternative examples, the shaft can be steerable. In such examples, the device can include one or more pull wires or other actuators for steering the shaft.

The shaft 106 can optionally include one or more radiopaque markers and/or echogenic markers (not shown), to enhance visualization of the position of the shaft 106.

Referring still to FIGS. 1 and 2, the RF puncture electrode 122 is joined to the shaft 106, and is positioned proud of the distal end 116 of the shaft 106. The RF puncture electrode 122 is electrically connectable to the RF generator 104 (as will be described below) and can deliver RF energy to tissue, to puncture the tissue. In the example shown, the RF puncture electrode 122 is atraumatic—that is, the RF puncture electrode 122 is blunt (e.g. rounded) in order to avoid damaging tissue unless RF energy is being delivered from the RF puncture electrode 122 to the tissue.

In some examples (not shown), the RF puncture electrode can be used to collect electrical signals from the heart.

Referring now to FIG. 3, in the example shown, the shaft 106 includes a polymeric sleeve 124 (e.g. a high-density polyethylene sleeve), and an electrical conductor 126 (also referred to herein as a ‘first electrical conductor’) in the form of a metallic hypotube (e.g. a stainless steel hypotube) received in the polymeric sleeve 124. The electrical conductor 126 extends from the proximal portion 110 to the distal portion 114. The electrical conductor 126 is electrically connectable to the RF generator 104 via the handle 108, and is electrically connected to the RF puncture electrode 122, so that RF energy can be delivered from the RF generator 104 to the RF puncture electrode 122 via the electrical conductor 126.

In an alternative example (not shown), the shaft can include a polymeric inner layer (e.g. a high-density polyethylene layer) and a polymeric outer layer (e.g. a low-density polyethylene layer), and the electrical conductor can be positioned between the polymeric inner layer and polymeric outer layer. In further alternative examples, the electrical conductor can be in the form of a wire or a braid instead of a hypotube.

Referring still to FIG. 3, in the example shown, the first electrical conductor 126 extends to a position shy of the distal end 116 of the shaft 106. A second electrical conductor 128 electrically connects the first electrical conductor 126 and the RF puncture electrode 122. The second electrical conductor 128 is in the form of a wire that has a first end that is joined to the first electrical conductor 126 and a second end that is spaced distally from the distal end 116 of the shaft 106 and is joined to the RF puncture electrode 122, to conduct RF energy from the first electrical conductor 126 to the RF puncture electrode 122.

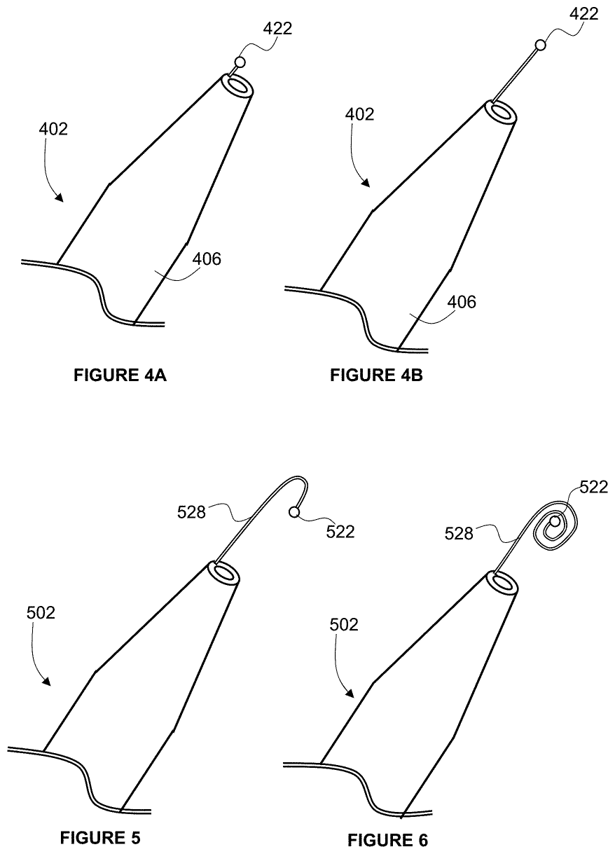

In the example shown, the RF puncture electrode 122 is generally fixed in position with respect to the shaft 106. An alternative example is shown in FIGS. 4A and 4B, in which features that are like those of FIGS. 1 to 3 are referenced with like reference characters, incremented by 300. In the medical puncture device 402 of FIGS. 4A and 4B, the RF puncture electrode 422 can be retracted towards the shaft 406 (as shown in FIG. 4A) and deployed away from the shaft 406 (as shown in FIG. 4B). In such examples, the device 402 can include one or more pull wires or other actuators (not shown) for retracting and deploying the RF puncture electrode 422. Another alternative example is shown in FIG. 5, in which features that are like those of FIGS. 1 to 3 are referenced with like reference characters, incremented by 400. In the medical puncture device 502 of FIG. 5, in addition to being retractable and deployable, the second electrical conductor 528 is J-shaped. This can enhance patient safety, as the electrode 522 will be directed back from tissue when deployed. Yet another alternative example is shown in FIGS. 6, in which features that are like those of FIGS. 1 to 3 are referenced with like reference characters, incremented by 500. In the medical puncture device 600 of FIG. 6, in addition to being retractable and deployable, the second electrical conductor 628 is coiled (also referred to as ‘pig-tail shaped’). Again, this can enhance patient safety, as the electrode 622 will be directed back from tissue when deployed. In yet further alternative examples (not shown), the electrode can be joined directly to the first electrical conductor, and the second electrical conductor can be omitted.

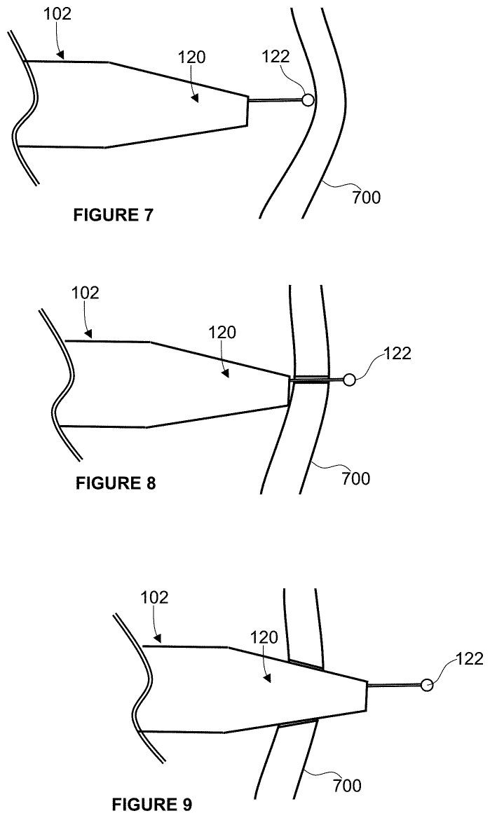

Referring now to FIGS. 7 to 9, a method for puncturing and dilating an atrial septum 700 using the medical puncture device 102 will be described. As a first step (not shown), a guidewire can be advanced into the superior vena cava, via the femoral vein. The medical puncture device 102 can then be advanced over the guidewire until the distal end 116 of the shaft is in the superior vena cava. The guidewire can then be removed. The medical puncture device 102 can then be pulled down into the right atrium, and positioned with the RF puncture electrode 122 against the fossa ovalis of the atrial septum 700, to tent the atrial septum 700, as shown in FIG. 7. Optionally, fluoroscopy or another visualization technique can be used to confirm the positioning of the medical puncture device 102. As a next step, as shown in FIG. 8, the RF generator 104 (not shown in FIG. 8) can be activated so that RF energy is delivered from the RF puncture electrode 122 to puncture the atrial septum 700 and pass the electrode 122 through the atrial septum 700 into the left atrium. Optionally fluoroscopy or another visualization technique can again be used to confirm the positioning of the electrode 122. As a next step, as shown in FIG. 9, the medical puncture device 102 can be advanced so that the dilating tip 120 passes through puncture in the atrial septum 700 and dilates the puncture. After the puncture has been dilated, various steps can be carried out (e.g. cryoablation, mitral valve replacement, or atrial appendage closure), depending on the nature of the medical procedure

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A medical puncture device comprising:
an elongate shaft having a proximal portion defining a proximal end and a distal portion defining a distal end, wherein the distal portion tapers in outer diameter going towards the distal end to define a dilating tip, wherein a lumen extends through the shaft from the proximal end to the distal end, and wherein the shaft comprises a first electrical conductor extending from the proximal portion to the distal portion and electrically connectable to a radiofrequency generator;
a radiofrequency puncture electrode positioned proud of the distal end and electrically connected to the first electrical conductor.

2. The medical puncture device of claim 1, wherein the shaft comprises a polymeric sleeve, and the first electrical conductor is in the form of a metallic hypotube received in the polymeric sleeve.

3. The medical puncture device of claim 2, wherein the polymeric sleeve is a high-density polyethylene sleeve and the metallic hypotube is a stainless steel hypotube.

4. The medical puncture device of claim 1, wherein the shaft comprises a polymeric inner layer and a polymeric outer layer, and the first electrical conductor is positioned between the polymeric inner layer and the polymeric outer layer.

5. The medial puncture device of claim 4, wherein the polymeric inner layer is a high-density polyethylene inner layer, and the polymeric outer layer is a low-density polyethylene layer.

6. The medical puncture device of claim 4, wherein the first electrical conductor is in the form of a wire or a braid.

7. The medical puncture device of claim 1, further comprising a second electrical conductor electrically connecting the electrode to the first electrical conductor.

8. The medical puncture device of claim 7, wherein
the second electrical conductor is in the form of a wire having a first end and a second end, wherein the first end is joined to the first electrical conductor, and the second end is spaced distally of the distal end and joined to the radiofrequency puncture electrode.

9. The medical puncture device of claim 8, wherein the wire is J-shaped or coiled.

10. The medical puncture device of claim 1, wherein the radiofrequency puncture electrode is retractable towards the shaft.

11. The medical puncture device of claim 1, wherein the radiofrequency puncture electrode is atraumatic.

12. The medical puncture device of claim 1, further comprising a handle at the proximal end of the shaft, wherein the first electrical conductor is electrically connectable to the radiofrequency generator via the handle.

13. The medical puncture device of claim 12, wherein the handle comprises a hemostatic valve.

14. The medical puncture device of claim 1, wherein the shaft has an outer diameter of between about 12.5 Fr and about 24 Fr.

15. The medical puncture device of claim 1, wherein the shaft is steerable.

16. The medical puncture device of claim 1, wherein the distal portion is curved.

17. The medical puncture device of claim 1, wherein the shaft comprises a radiopaque marker.

18. The medical puncture device of claim 1, wherein the shaft comprises an echogenic marker.

19. A medical method comprising:
a. advancing towards an atrial septum a medical puncture device comprising:
an elongate shaft having a proximal portion defining a proximal end and a distal portion defining a distal end, wherein the distal portion tapers in outer diameter going towards the distal end to define a dilating tip, wherein a lumen extends through the shaft from the proximal end to the distal end, and wherein the shaft comprises a first electrical conductor extending from the proximal portion to the distal portion and electrically connectable to a radiofrequency generator; and
a radiofrequency puncture electrode positioned proud of the distal end and electrically connected to the first electrical conductor;
b. delivering radiofrequency energy from the radiofrequency puncture electrode to create a puncture in the atrial septum; and
c. advancing the dilating tip through the puncture to dilate the puncture.

20. A medical puncture system comprising:
a radiofrequency generator; and
a medical puncture device comprising i) an elongate shaft having a proximal portion defining a proximal end and a distal portion defining a distal end, wherein the distal portion tapers in outer diameter going towards the distal end to define a dilating tip, wherein a lumen extends through the shaft from the proximal end to the distal end, and wherein the shaft comprises a first electrical conductor extending from the proximal portion to the distal portion and electrically connectable to a radiofrequency generator; and
a radiofrequency puncture electrode positioned proud of the distal end and electrically connected to the first electrical conductor.
